(19) United States
(12) Patent Application Publication
(10) Pub. No.: US 2007/0042487 A1
(43) Pub. Date: Feb. 22, 2007

(54) Bioreactor valve island

(75) Inventor: Mark Cheshire, Tividale (GB)

Correspondence Address: THE OLLILA LAW GROUP LLC, 2060 BROADWAY, SUITE 300, BOULDER, CO, 80302, US

(73) Assignee: IMI Norgren, Inc., Littleton, CO (US)

(21) Appl. No.: 11/207,391

(22) Filed: Aug. 19, 2005

(51) Int. Cl. C12M 1/00; C12M 1/36

(52) U.S. Cl. 435/283.1; 435/289.1; 435/286.6

(57) ABSTRACT

A bioreactor valve island is provided according to an embodiment of the invention. The bioreactor valve island includes a sparge mixing region that receives and mixes a first air input and one or more first gas inputs and an overlay mixing region that receives and mixes a second air input and one or more second gas inputs. The bioreactor valve island further includes one or more output valve pairs. An output valve pair of the one or more output valve pairs receives a gas from a gas supply of one or more corresponding gas supplies and selectively outputs both a first gas input of the gas to the sparge mixing region and a second gas input of the gas to the overlay mixing region.

### BACKGROUND OF THE INVENTION

1. Field of the Invention

The present invention relates to a bioreactor valve island.

2. Statement of the Problem

A bioreactor module is a device that provides specific environmental conditions in which to grow some manner of organic or living material. For example, a bioreactor module can be employed for growing plant or animal cells, bacteria, etc. As such, a bioreactor module is a vessel that is designed to achieve and maintain specific growing conditions.

One environmental condition of a bioreactor module that must be carefully controlled is the introduction and presence of gas mixtures. The gas mixture must often comprise specific proportions of designated gases for growing living organisms. For example, it is common in a bioreactor module to introduce a mixture of air, oxygen, carbon dioxide, and nitrogen into both an overlay input and a sparge input of the bioreactor module. The bioreactor overlay component passes an overlay gas mixture over the biological material being grown in the bioreactor module. The bioreactor sparge component passes a sparge gas mixture through the biological material.

In the prior art, delivery of such gases has commonly been performed by a plurality of valves. The plurality of prior art valves are connected to a plurality of air and gas sources. Each air and gas source may be connected to at least two valves. The prior art therefore typically requires a plethora of conduits and connections. Each prior art bioreactor valve is further connected to the bioreactor module by one or more corresponding conduits and connectors. This is unnecessarily costly and complicated.

### SUMMARY OF THE SOLUTION

A bioreactor valve island is provided according to an embodiment of the invention. The bioreactor valve island comprises a sparge mixing region that receives and mixes a first air input and one or more first gas inputs and an overlay mixing region that receives and mixes a second air input and one or more second gas inputs. The bioreactor valve island further comprises one or more output valve pairs. An output valve pair of the one or more output valve pairs receives a gas from a gas supply of one or more corresponding gas supplies and selectively outputs both a first gas input of the gas to the sparge mixing region and a second gas input of the gas to the overlay mixing region.

A bioreactor valve island is provided according to an embodiment of the invention. The bioreactor valve island comprises a sparge mixing region that receives and mixes a first air input and one or more first gas inputs and an overlay mixing region that receives and mixes a second air input and one or more second gas inputs. The bioreactor valve island further comprises one or more output valve pairs. An output valve pair of the one or more output valve pairs receives a gas from a gas supply of one or more corresponding gas supplies and selectively outputs both a first gas input of the gas to the sparge mixing region and a second gas input of the gas to the overlay mixing region. The bioreactor valve island further comprises two air input valves in communication with the sparge mixing region and the overlay mixing region. A first air input valve of the two air input valves selectively inputs air to the sparge mixing region and a second air input valve selectively inputs air to the overlay mixing region. The bioreactor valve island further comprises one or more gas input valves in communication with the one or more output valve pairs. A gas input valve of the one or more gas input valves selectively inputs a gas to a corresponding output valve pair.

A method of providing a mixed sparge supply and a mixed overlay supply for a bioreactor module from a bioreactor valve island is provided according to an embodiment of the invention. The method comprises selectively outputting one or more first gas inputs to a sparge mixing region of the bioreactor valve island, selectively outputting one or more second gas inputs to an overlay mixing region of the bioreactor valve island, mixing a first air input and the one or more first gas inputs in the sparge manifold mixer of the bioreactor valve island in order to obtain the mixed sparge supply, and mixing a second air input and the one or more second gas inputs in the overlay manifold mixer of the bioreactor valve island in order to obtain the mixed overlay supply.

### ASPECTS OF THE INVENTION

In one embodiment of the bioreactor valve island, a gas input of the one or more first gas inputs and the one or more second gas inputs comprises an oxygen input.

In another embodiment of the bioreactor valve island, a gas input of the one or more first gas inputs and the one or more second gas inputs comprises a carbon dioxide input.

In yet another embodiment of the bioreactor valve island, a gas input of the one or more first gas inputs and the one or more second gas inputs comprises a nitrogen input.

In yet another embodiment of the bioreactor valve island, an air input of first air input and the second air input comprises a metered air input.

In yet another embodiment of the bioreactor valve island, a gas input of the one or more first gas inputs and the one or more second gas inputs comprises a metered gas input.

In yet another embodiment of the bioreactor valve island, the one or more output valve pairs are adapted to be electronically controlled.

In yet another embodiment of the bioreactor valve island, the bioreactor valve island further comprises two air input valves in communication with the sparge mixing region and the overlay mixing region, with the two air input valves selectively inputting air to the sparge mixing region and to the overlay mixing region, and one or more gas input valves in communication with the one or more output valve pairs, with a gas input valve of the one or more gas input valves selectively inputting a gas to a corresponding output valve pair.

In yet another embodiment of the bioreactor valve island, the bioreactor valve island further comprises two air flow meters positioned between the two air input valves and the sparge mixing region and the overlay mixing region, with the two air flow meters metering the air being supplied to the sparge mixing region and to the overlay mixing region, and one or more gas flow meters positioned between the one or more gas input valves and the one or more output valve pairs, with the one or more gas flow meters metering the one or more gases being supplied to the sparge mixing region and to the overlay mixing region.

In one embodiment of the method, a gas input of the one or more first gas inputs and the one or more second gas inputs comprises an oxygen input.

In another embodiment of the method, a gas input of the one or more first gas inputs and the one or more second gas inputs comprises a carbon dioxide input.

In yet another embodiment of the method, a gas input of the one or more first gas inputs and the one or more second gas inputs comprises a nitrogen input.

In yet another embodiment of the method, an air input of first air input and the second air input comprises a metered air input.

In yet another embodiment of the method, a gas input of the one or more first gas inputs and the one or more second gas inputs comprises a metered gas input.

In yet another embodiment of the method, the selectively outputting the one or more first gas inputs and the one or more second gas inputs comprises actuating electronically controlled valves in a bioreactor valve island.

In yet another embodiment of the method, the method further comprises metering each first gas input of the one or more first gas inputs, metering each second gas input of the one or more second gas inputs, metering the first air input, and metering the second air input.

### DESCRIPTION OF THE DRAWINGS

The same reference number represents the same element on all drawings. It should be understood that the drawings are not necessarily to scale.

FIG. 1 shows a bioreactor valve island according to an embodiment of the invention.

FIG. 2 shows the bioreactor valve island according to an embodiment of the invention.

FIG. 3 shows a bioreactor valve island implementation according to the schematic of FIG. 2.

FIG. 4 shows a bioreactor system according to an embodiment of the invention.

FIG. 5 shows the bioreactor valve island according to an embodiment of the invention.

### DETAILED DESCRIPTION OF THE INVENTION

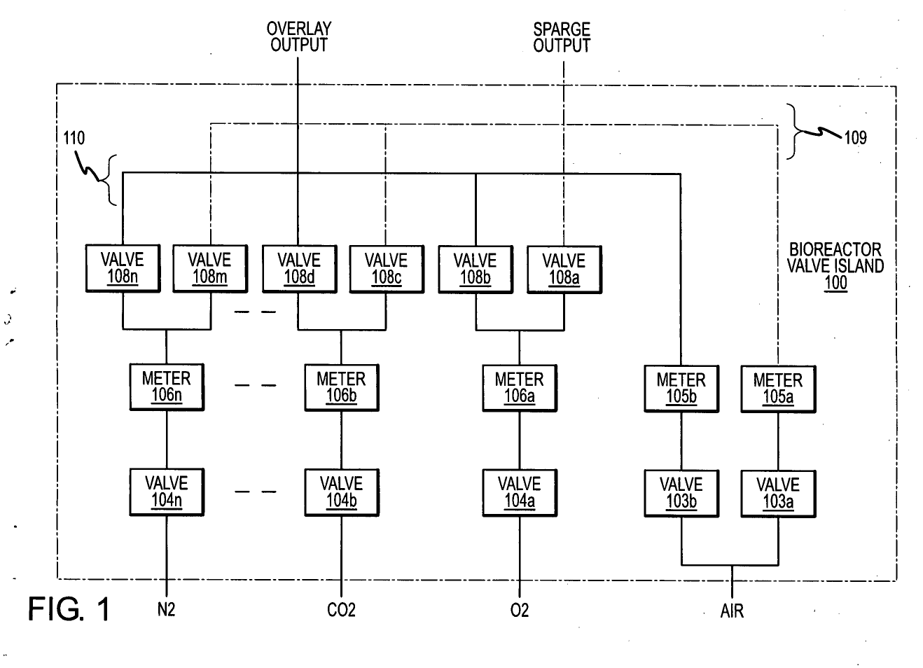

FIGS. 1-5 and the following description depict specific examples to teach those skilled in the art how to make and use the best mode of the invention. For the purpose of teaching inventive principles, some conventional aspects have been simplified or omitted. Those skilled in the art will appreciate variations from these examples that fall within the scope of the invention. Those skilled in the art will appreciate that the features described below can be combined in various ways to form multiple variations of the invention. As a result, the invention is not limited to the specific examples described below, but only by the claims and their equivalents.

FIG. 1 shows a bioreactor valve island 100 according to an embodiment of the invention. The bioreactor valve island 100 in this embodiment includes air input valves 103, gas input valves 104, air flow meters 105, gas flow meters 106, output valve pairs 108, a sparge mixing region 109, and an overlay mixing region 110.

The air input valves 103 in this embodiment comprise a first air input valve 103a and a second air input valve 103b. The air input valves 103 selectively input air for both the sparge mixing region 109 and the overlay mixing region 110. The air input valves 103 can be selectively opened and closed in order to admit desired volumes of air to both mixing manifolds. The air input valves 103a-103b can therefore select and provide air to one or both mixing manifolds 109 and 110. The air input valves 103a-103b can be actuated by some manner of controller or other external device (not shown).

The gas input valves 104 can comprise a plurality of gas input valves 104a-104n that receive gas inputs. The number of gas input valves 104 can be varied according to the number of gases to be used. In the figure shown, the gas input valves 104 comprise three gas input valves. For example, the three input valves 104 can admit oxygen (OXY), carbon dioxide (CO₂), and nitrogen (N). However, it should be understood that the number of gas inputs can be varied as desired and various gases can be inputted into the bioreactor valve island 100.

The air flow meters 105a and 105b measure air flows received from the air input valves 103a and 103b. The air flow meters 105a and 105b in one embodiment communicate air flow measurement signals to the controller or other external device. The air flow measurement signals can be used to control the air input valves 103a and 103b and regulate the flow of air into the bioreactor valve island 100.

The gas flow meters 106a-106n measure flows of each gas received from the gas input valves 104a-104n. The gas flow meters 106a-106n therefore correspond in number to the gas input valves 104a-104n. The gas flow meters 106a-106n in one embodiment communicate gas flow measurement signals to the controller or other external device, wherein the gas flow measurement signals can be used to control the gas input valves 104 and regulate the flow of gases into the bioreactor valve island 100. In this manner, the volumes of gases delivered to both a sparge and an overlay of a bioreactor module can be precisely controlled.

The output valves 108A-108N regulate the output of gases to the sparge mixing region 109 and the overlay mixing region 110. The output valves 108A-108N can therefore select one or both mixing manifolds 109 and 110 to receive the gases in varying amounts. The output valves 108A-108N can deliver gases to the sparge mixing region 109 and the overlay mixing region 100 in different proportions, if desired.

The sparge mixing region 109 receives predetermined volumes of air and gases, mixes them, and outputs them in a sparge output. The sparge output can be connected to a sparge input of the bioreactor module. The sparge output of mixed air and gas can therefore be used to bubble through and sparge a biological fluid in order to enable and enhance growth of the biological material.

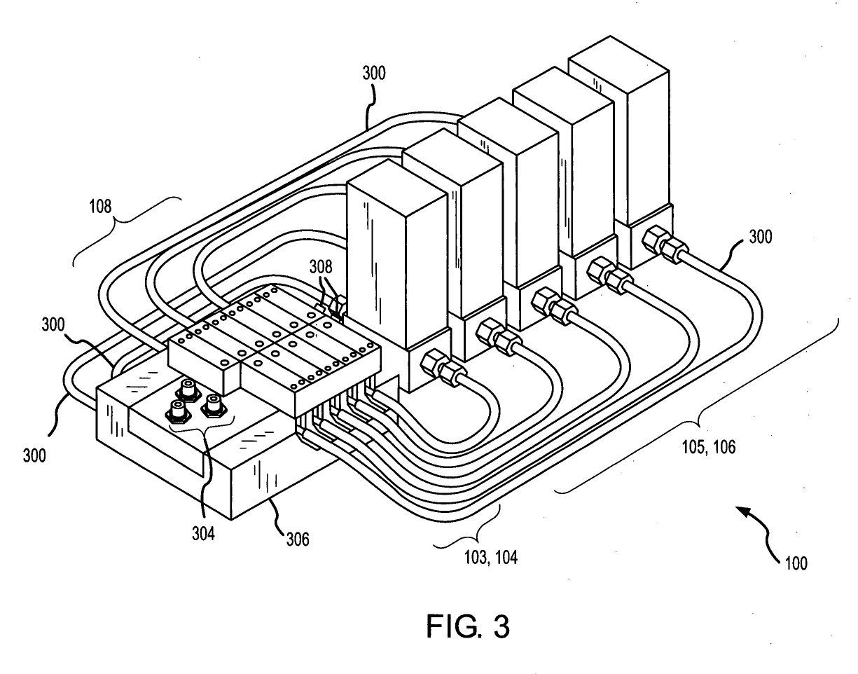

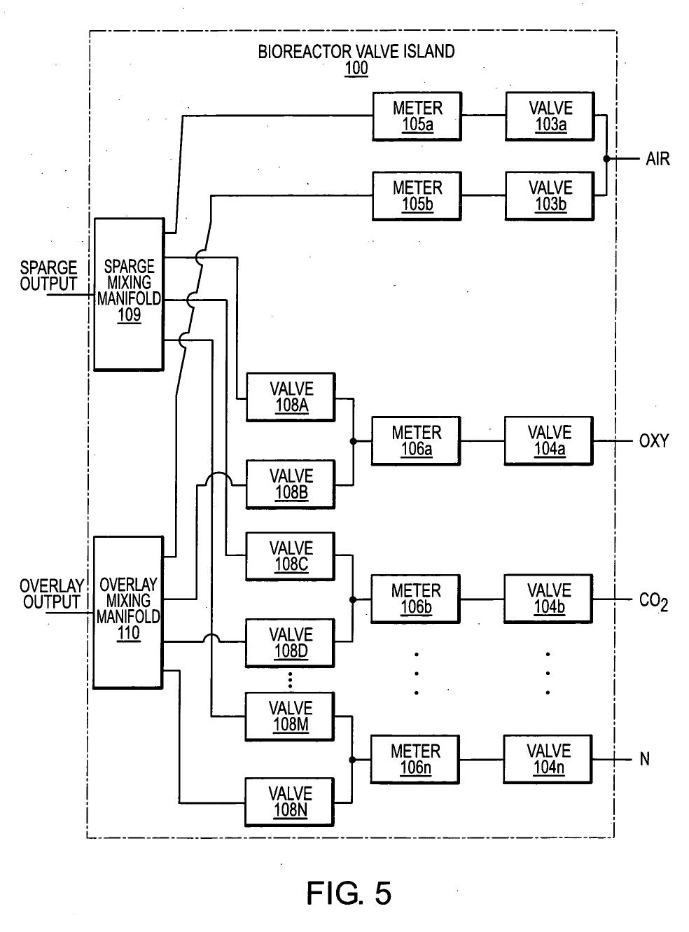

The sparge mixing region 109 can comprise a plurality of conduits that feed into and join a single output conduit, i.e., the output to the bioreactor module sparge, as shown in the figure. The sparge mixing region 109 can therefore mix the air and gas supplied by the various valves. The conduits can comprise bores formed in a base 306 of the bioreactor valve island 100 (see FIG. 3). Alternatively, the conduits can comprise a manifold component that is integrated into the bioreactor valve island 100 (see FIG. 5), i.e., the sparge mixing region 109 can comprise a sparge mixing manifold 109.

The overlay mixing region 110 receives predetermined volumes of air and gases, mixes them, and outputs them in an overlay output. The overlay mixing region 110 can receive the same or different volumes of gases than the sparge mixing region 109. The overlay mixing region 110 can receive the same or different types of gases than the sparge mixing region 109. The overlay output can be connected to an overlay input of the bioreactor module. The overlay output of mixed air and gas can therefore be used to flow over the biological fluid/material in the bioreactor module.

The overlay mixing region 110 can comprise a plurality of conduits that feed into and join a single output conduit, i.e., the output to the bioreactor module overlay, as shown in the figure. The overlay mixing region 110 can therefore mix the air and gas supplied by the various valves. As discussed for the sparge mixing region 109, the conduits can comprise bores formed in a base 306 of the bioreactor valve island 100 or the overlay mixing region 110 can comprise a manifold component that is integrated into the bioreactor valve island 100.

The bioreactor valve island 100 in the embodiment shown includes the input valves 103 and 104, the output valves 108, the sparge mixing region 109, and the overlay mixing region 110. In addition, in this embodiment the flow meters 105a-105b and 106a-106n are included in the bioreactor valve island 100. Consequently, the air and gas streams from the air input valves 103a-103b and from the gas input valves 104a-104n remain in the bioreactor valve island 100.

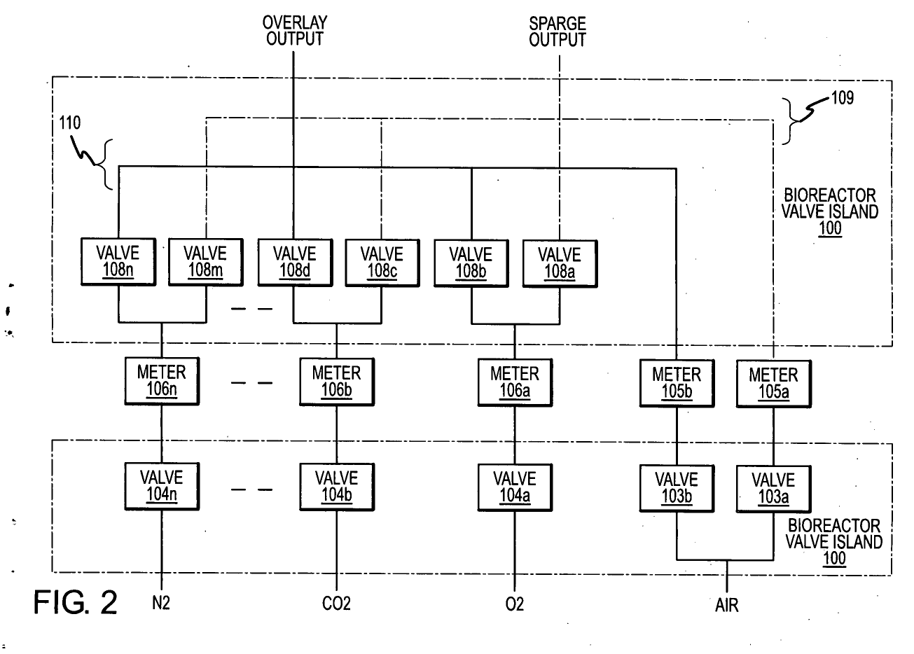

FIG. 2 shows the bioreactor valve island 100 according to an embodiment of the invention. In this embodiment, the flow meters 105a-105b and 106a-106n are external devices outside the bioreactor valve island 100. Consequently, the air and gas streams from the air input valves 103a-103b and from the gas input valves 104a-104n are conducted to the external flow meters and then returned.

FIG. 3 shows a bioreactor valve island 100 implementation according to the schematic of FIG. 2. In this embodiment, the flow meters 105 and 106 are external devices that are connected to the bioreactor valve island 100 by conduits 300. The bioreactor valve island 100 can further include a base 306 and one or more communication ports 304 that exchange communication signals with external devices. The one or more communication ports 304 can be connected to any manner of communication or instrumentation bus. The one or more communication ports 304 enable the valves of the bioreactor valve island 100 to be electronically controlled. This can include the air input valves 103a-103b, the gas input valves 104a-104n, and the output valve pairs 108A-108M.

The bioreactor valve island 100 can further include indicators 308 formed as part of each valve 103, 104, and 108. The indicators 308 can give visual indications of valve actuation. In one embodiment, the indicators 308 can comprise LEDs that are lit during valve actuation, for example. However, it should be understood that any manner of indicator can be employed.

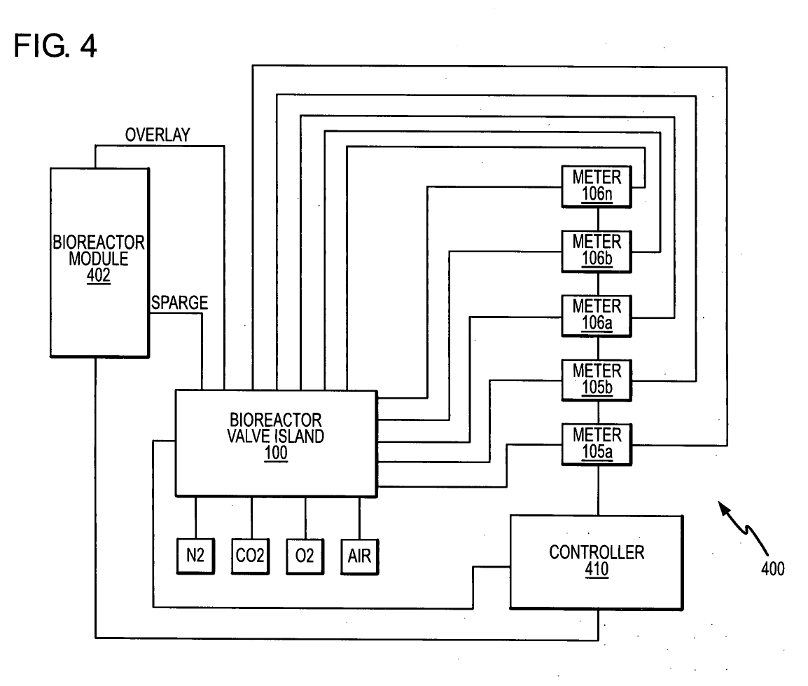

FIG. 4 shows a bioreactor system 400 according to an embodiment of the invention. The bioreactor system 400 in this embodiment includes a bioreactor module 402, the bioreactor valve island 100, the air flow meters 105a-105b, the gas flow meters 106a-106n, and a controller 410. The controller 410 can be connected to each of these components by some manner of bus or cable. For example, the components can be interconnected by an instrumentation or communication bus, such as DEVICENET, FIELDBUS, etc.

The controller 410 can comprise any manner of computer, processor, controller, or control circuitry. The controller 410 can include appropriate software and control routines, as necessary. The controller 410 controls the application of air and gases to the bioreactor module 401, among other things. The controller 410 can control the types of gases selected for provision to the bioreactor module 402. The controller 410 can control the quantities of air and gases provided to the bioreactor module 402. The controller 410 can control the ratios or percentages of air and gases provided to the bioreactor module 402. The controller 410 can control the provision of air and gases to each of the sparge and the overlay.

The controller 410 can communicate with the bioreactor module 402 and can send commands to and receive data from the bioreactor module 402. The controller 410 can communicate with the bioreactor valve island 100. The controller 410 can send valve commands to individual valves or valve groupings. The controller 410 can receive valve actuation data from the bioreactor valve island 100 and can control the bioreactor valve island 100 based on the valve actuation data. The controller 410 can communicate with the air flow meters 105a-105b and with the gas flow meters 106a-106n. The controller 410 can receive flow data from the flow meters, such as mass flow rate data and density data. The controller 410 can process the flow data and can use the flow data to control the bioreactor valve island 100.

FIG. 5 shows the bioreactor valve island 100 according to an embodiment of the invention. In this embodiment, the sparge mixing region 109 comprises a sparge mixing manifold 109 and the overlay mixing region 110 comprises an overlay mixing manifold 110. The manifolds can comprise components that are integrated into the bioreactor valve island 100, for example. The sparge mixing region 109 and the overlay mixing region 110 can alternatively comprise a single manifold component that includes multiple inputs and two unique outputs.

The bioreactor valve island according to the invention can be employed according to any of the embodiments in order to provide several advantages, if desired. The bioreactor valve island according to the invention offers a comprehensive valve island that delivers mixed gases to a bioreactor module. The bioreactor valve island according to the invention offers a comprehensive valve island that delivers metered gases to a bioreactor module. The bioreactor valve island according to the invention generates gas mixtures for both the sparge portion and the overlay portion of a bioreactor. The bioreactor valve island according to the invention requires a minimum of input connections and output connections. The bioreactor valve island according to the invention can be fully electronically controlled.

We claim:

1. A bioreactor valve island comprising:
a sparge mixing region that receives and mixes a first air input and one or more first gas inputs;
an overlay mixing region that receives and mixes a second air input and one or more second gas inputs; and
one or more output valve pairs, with an output valve pair of the one or more output valve pairs receiving a gas from a gas supply of one or more corresponding gas supplies and selectively outputting both a first gas input of the gas to the sparge mixing region and a second gas input of the gas to the overlay mixing region.

2. The bioreactor valve island of claim 1, with a gas input of the one or more first gas inputs and the one or more second gas inputs comprising an oxygen input.

3. The bioreactor valve island of claim 1, with a gas input of the one or more first gas inputs and the one or more second gas inputs comprising a carbon dioxide input.

4. The bioreactor valve island of claim 1, with a gas input of the one or more first gas inputs and the one or more second gas inputs comprising a nitrogen input.

5. The bioreactor valve island of claim 1, with an air input of first air input and the second air input comprising a metered air input.

6. The bioreactor valve island of claim 1, with a gas input of the one or more first gas inputs and the one or more second gas inputs comprising a metered gas input.

7. The bioreactor valve island of claim 1, with the one or more output valve pairs (108) being adapted to be electronically controlled.

8. The bioreactor valve island of claim 1, further comprising:
two air input valves in communication with the sparge mixing region and the overlay mixing region, with the two air input valves selectively inputting air to the sparge mixing region and to the overlay mixing region; and
one or more gas input valves in communication with the one or more output valve pairs, with a gas input valve of the one or more gas input valves selectively inputting a gas to a corresponding output valve pair.

9. The bioreactor valve island of claim 8, further comprising:
two air flow meters positioned between the two air input valves and the sparge mixing region and the overlay mixing region, with the two air flow meters metering the air being supplied to the sparge mixing region and to the overlay mixing region; and
one or more gas flow meters positioned between the one or more gas input valves and the one or more output valve pairs, with the one or more gas flow meters metering the one or more gases being supplied to the sparge mixing region and to the overlay mixing region.

10. A bioreactor valve island comprising:
a sparge mixing region that receives and mixes a first air input and one or more first gas inputs;
an overlay mixing region that receives and mixes a second air input and one or mote second gas inputs;
one or more output valve pairs, with an output valve pair of the one or more output valve pairs receiving a gas from a gas supply of one or more corresponding gas supplies and selectively outputting both a first gas input of the gas to the sparge mixing region and a second gas input of the gas to the overlay mixing region;
two air input valves in communication with the sparge mixing region and the overlay mixing region, with a first air input valve of the two air input valves selectively inputting air to the sparge mixing region and with a second air input valve selectively inputting air to the overlay mixing region; and
one or more gas input valves in communication with the one or more output valve pairs, with a gas input valve of the one or more gas input valves selectively inputting a gas to a corresponding output valve pair.

11. The bioreactor valve island of claim 10, with a gas input of the one or more first gas inputs and the one or more second gas inputs comprising an oxygen input.

12. The bioreactor valve island of claim 10, with a gas input of the one or more first gas inputs and the one or more second gas inputs comprising a carbon dioxide input.

13. The bioreactor valve island of claim 10, with a gas input of the one or more first gas inputs and the one or more second gas inputs comprising a nitrogen input.

14. The bioreactor valve island of claim 10, with an air input of first air input and the second air input comprising a metered air input.

15. The bioreactor valve island of claim 10, with a gas input of the one or more first gas inputs and the one or more second gas inputs comprising a metered gas input.

16. The bioreactor valve island of claim 10, with the one or more output valve pairs being adapted to be electronically controlled.

17. The bioreactor valve island of claim 10, with the one or more output valve pairs, the two air input valves, and the one or more gas input valves being adapted to be electronically controlled.

18. The bioreactor valve island of claim 10, further comprising:
two air flow meters positioned between the two air input valves and the sparge mixing region and the overlay mixing region, with the two air flow meters metering the air being supplied to the sparge mixing region and to the overlay mixing region; and
one or more gas flow meters positioned between the one or more gas input valves and the one or more output valve pairs, with the one or more gas flow meters metering the one or more gases being supplied to the sparge mixing region and to the overlay mixing region.

19. A method of providing a mixed sparge supply and a mixed overlay supply for a bioreactor module from a bioreactor valve island, the method comprising:
selectively outputting one or more first gas inputs to a sparge mixing region of the bioreactor valve island;
selectively outputting one or more second gas inputs to an overlay mixing region of the bioreactor valve island;
mixing a first air input and the one or more first gas inputs in the sparge manifold mixer of the bioreactor valve island in order to obtain the mixed sparge supply; and
mixing a second air input and the one or more second gas inputs in the overlay manifold mixer of the bioreactor valve island in order to obtain the mixed overlay supply.

20. The method of claim 19, with a gas input of the one or more first gas inputs and the one or more second gas inputs comprising an oxygen input.

21. The method of claim 19, with a gas input of the one or more first gas inputs and the one or more second gas inputs comprising a carbon dioxide input.

22. The method of claim 19, with a gas input of the one or more first gas inputs and the one or more second gas inputs comprising a nitrogen input.

23. The method of claim 19, with an air input of first air input and the second air input comprising a metered air input.

24. The method of claim 19, with a gas input of the one or more first gas inputs and the one or more second gas inputs comprising a metered gas input.

25. The method of claim 19, with the selectively outputting the one or more first gas inputs and the one or more second gas inputs comprising actuating electronically controlled valves in a bioreactor valve island.

26. The method of claim 19, further comprising:
metering each first gas input of the one or more first gas inputs;
metering each second gas input of the one or more second gas inputs;
metering the first air input; and
metering the second air input.
